(19) United States
(12) Patent Application Publication
(10) Pub. No.: US 2024/0082432 A1
(43) Pub. Date: Mar. 14, 2024

(54) COMPOSITION AND USE THEREOF

(71) Applicant: Debin CUI, Liaoyang (CN)

(72) Inventor: Debin CUI, Liaoyang (CN)

(21) Appl. No.: 18/275,424

(22) PCT Filed: Jan. 28, 2022
(86) PCT No.: PCT/CN2022/074766

§ 371 Date: Aug. 2, 2023

(30) Foreign Application Priority Data
Feb. 5, 2021 (CN) 202110166856.6

(51) Int. Cl. A61K 49/00; A61K 31/045; A61K 31/19; A61K 31/7004

(52) U.S. Cl. CPC A61K 49/0008; A61K 31/045; A61K 31/19; A61K 31/7004; A01K 67/00

(57) ABSTRACT

A composition and a use thereof are provided. The composition includes a saccharide and an acid or a saccharide, an acid, and an alcohol. The composition may be used to prepare animal models simulating various human diseases, and thus used for prevention and treatment of human diseases and screening of drugs.

### CROSS REFERENCE TO THE RELATED APPLICATIONS

This application is the national phase entry of International Application No. PCT/CN2022/074766, filed on Jan. 28, 2022, which is based upon and claims priority to Chinese Patent Application No. 202110166856.6, filed on Feb. 5, 2021, the entire contents of which are incorporated herein by reference.

### TECHNICAL FIELD

The present disclosure belongs to the technical field of life science, and relates to a composition and a use thereof.

### BACKGROUND

The understanding of humans' diseases is continuously improved with social developments and scientific and technological progress. Many existing major diseases and chronic diseases are almost incurable, most of which can only be maintained, alleviated, and controlled to progress slowly. Globally, cancer, cardiovascular diseases (CVDs), diabetes, and the like are the most fatal and most common diseases in addition to infectious diseases. Medical and scientific researchers are actively working to break through the existing technologies and pursue the early achievement of completely solving these major and chronic diseases endangering human life. In the past hundred years, although the research and understanding of various diseases have been continuously improved, a major key problem has not been solved, that is, etiology is unclear, which makes only a symptomatic treatment available. If treatment is conducted from the perspective of etiology, these diseases will be quickly cured. Therefore, only when the pathogenesis of a disease is clarified, the disease can be effectively cured. Cancer, CVDs, and diabetes are common diseases with the highest incidence rate worldwide, and due to such a high incidence rate, the etiology of these diseases may be clarified based on laws, which lays a foundation for drug screening and clinical research.

In order to meet the needs of drug screening and clinical practice, accurately preparing an animal disease model that completely simulates normal human eating habits is key to determining the success of drug screening and plays an important role in the prevention and cure of these diseases in humans.

### SUMMARY

In order to solve the problems existing in the prior art, the present disclosure provides a composition and a use thereof. The composition of the present disclosure may be used to prepare animal models simulating various human diseases, and thus used for prevention and treatment of human diseases and screening of drugs. The composition of the present disclosure includes a saccharide and an acid or a saccharide, an acid, and an alcohol; and the model animal is not limited to a mammal. The composition of the present disclosure has a great value for prevention and treatment of various human diseases such as diabetes, cancer, CVDs, kidney diseases, and liver diseases.

The present disclosure is implemented by the following technical solutions:

In a first aspect, the present disclosure provides a composition, including a saccharide and an acid.

Preferably, the saccharide is a polyhydroxy (two or more hydroxy groups) aldehyde or ketone compound, and the saccharide is composed of carbon, hydrogen, and oxygen elements and is preferably glucose.

Preferably, the acid is an organic acid or an inorganic acid.

Preferably, the acid is selected from the group consisting of 85% (mass percentage concentration) phosphoric acid, 37% (mass percentage concentration) hydrochloric acid, 99.5% (mass percentage concentration) acetic acid, and 85% (mass percentage concentration) lactic acid.

Preferably, a mass-to-volume ratio of the saccharide to the acid is 1:100 to 100:1 (g/mL), preferably 1:20 to 20:1 (g/mL), and more preferably 1:0.375 (g/mL) or 5:1 (g/mL).

Preferably, the composition has a pH of lower than 4 and preferably lower than 3.22.

Preferably, the composition is a food composition.

In a specific embodiment, the composition is: a mixture of 1 g of glucose and 0.375 mL of hydrochloric acid (mass percentage concentration: 37%);
a mixed solution of a glucose injection (mass percentage concentration: 50%) and acetic acid (mass percentage concentration: 99.5%) (in a volume ratio of 10:1);
a mixed solution of a glucose injection (mass percentage concentration: 50%) and acetic acid (mass percentage concentration: 99.5%) (in a volume ratio of 20:1);
a mixed solution of a glucose injection (mass percentage concentration: 50%) and lactic acid (mass percentage concentration: 85%) (in a volume ratio of 10:1); or
a mixed solution of a glucose injection (mass percentage concentration: 50%) and phosphoric acid (mass percentage concentration: 85%) (in a volume ratio of 10:1).

Preferably, the composition further includes an alcohol, that is, the composition includes the saccharide, the acid, and the alcohol.

Preferably, the alcohol is ethanol.

Preferably, the saccharide, the acid, and the alcohol are in a ratio of 1:100:1 to 100:1:100 (g/mL/mL), preferably 1:20:1 to 20:1:20 (g/mL/mL), and more preferably 10:5:5 (g/mL/mL).

Preferably, the composition is a food composition, and in the present disclosure, the food composition simulates a food that ingested by a natural person in a normal diet.

In a second aspect, the present disclosure provides a preparation method of the composition, including: mixing the saccharide and the acid or the saccharide, the acid, and the alcohol according to the above ratio.

In a third aspect, the present disclosure provides a use of the composition in preparation of an animal model. For example, the composition may induce cancer (such as lung cancer) and thus can serve as an inducer for preparation of a cancer model.

Preferably, a model animal used for the animal model includes, but is not limited to, a mammal, such as a mouse, a rat, a rabbit, a dog, a monkey, a cat, a chicken, or a pig.

Preferably, the animal model is prepared by administering the composition to the model animal through an administration route such as feeding, intragastric administration, intravenous drip, intravenous injection, or skin penetration.

Preferably, the animal model includes, but is not limited to, an animal model of diabetes, cancer, AS or a CVD caused thereby, or another disease.

Preferably, the animal model is used in drug screening for prevention and/or treatment of cancer, diabetes, AS or a CVD caused thereby, or another disease.

In a fourth aspect, the present disclosure provides a use of an acid in preparation of an animal model. For example, the acid may induce cancer (such as lung cancer) and thus can serve as an inducer for preparation of a cancer model.

Preferably, the acid is an inorganic acid (such as hydrochloric acid) or an organic acid; preferably, the acid is selected from the group consisting of 13.88% (mass percentage concentration) hydrochloric acid, 7.73% (mass percentage concentration) lactic acid, 4.74% to 9.05% (mass percentage concentration) acetic acid, and 7.73% (mass percentage concentration) phosphoric acid; and
preferably, the acid has a pH of lower than 3.22.

In a specific embodiment, the acid is:
a mixed solution of normal saline (NS) and concentrated hydrochloric acid (mass percentage concentration: 37%) (in a volume ratio of 5:3);
a mixed solution of NS and acetic acid (mass percentage concentration: 99.5%) (in a volume ratio of 10:1);
a mixed solution of NS and lactic acid (mass percentage concentration: 85%) (in a volume ratio of 10:1);
a mixed solution of NS and phosphoric acid (mass percentage concentration: 85%) (in a volume ratio of 10:1);
a mixed solution of NS (10 mL) and acetic acid (mass percentage concentration: 99.5%, 1 mL);
a mixed solution of NS (10 mL) and acetic acid (mass percentage concentration: 99.5%, 0.5 mL);
a mixed solution of NS (10 mL) and lactic acid (mass percentage concentration: 85%, 1 mL);
a mixed solution of NS (10 mL) and phosphoric acid (mass percentage concentration: 85%, 1 mL); or
a mixed solution of NS (2.5 mL) and hydrochloric acid (mass percentage concentration: 37%, 1.5 mL).

Preferably, a model animal used for the animal model includes, but is not limited to, a mammal, such as a mouse, a rat, a rabbit, a dog, a monkey, a cat, a chicken, or a pig,
preferably, the animal model is prepared by administering the composition to the model animal through an administration route such as feeding, intragastric administration, intravenous drip, intravenous injection, or skin penetration; and
preferably, the animal model includes, but is not limited to, an animal model of diabetes, cancer, AS or a CVD caused thereby, or another disease.

Preferably, the animal model is used in drug screening for prevention and/or treatment of cancer, diabetes, AS or a CVD caused thereby, or another disease.

In a fifth aspect, the present disclosure provides a modeling method, including: administering the composition or the acid described above to a model animal,
where preferably, the model animal includes, but is not limited to, a mammal, such as a mouse, a rat, a rabbit, a dog, a monkey, a cat, a chicken, or a pig;
preferably, a prepared model includes, but is not limited to, an animal model of diabetes, cancer (such as lung cancer), AS or a CVD caused thereby, or another disease;
preferably, the acid is an inorganic acid (such as hydrochloric acid) or an organic acid;
preferably, the acid is selected from the group consisting of 13.88% (mass percentage concentration) hydrochloric acid, 7.73% (mass percentage concentration) lactic acid, 4.74% to 9.05% (mass percentage concentration) acetic acid, and 7.73% (mass percentage concentration) phosphoric acid; and
preferably, the acid has a pH of lower than 4 and preferably lower than 3.22.

Compared with the prior art, the present disclosure has the following beneficial technical effects:
(1) The composition of the present disclosure can be used in preparation of an animal model to provide a theoretical basis for prevention and treatment of major diseases in humans.
(2) The composition of the present disclosure can be used to prepare an animal disease model completely simulating normal human eating habits without other chemicals, and is suitable for preparation of animal models of a variety of human diseases.
(3) The composition of the present disclosure is an accurate animal model for screening of a human and/or animal drug.

Studies inside and outside China have shown that inflammation can induce cancer. In the present disclosure, the saccharide and acid are necessary for the existence of all animal organisms. It is undeniable that, in addition to meeting normal metabolic needs of a body, the saccharide and acid will also be partly used for generation of an aldehyde compound attached to an outer membrane of a cell after entering the body; and when a tissue undergoes inflammation and the aldehyde compound attached to the outer membrane of the cell reaches a threshold, there will be cell necrosis at an inflammation site, and after the cell necrosis, the aldehyde compound will accumulate, which aggravates the cell necrosis and the infiltration in normal tissue cells to gradually produce necrotic spots, nodules, small lumps, and tumors. When the composition of the present disclosure is used for modeling, it can be seen that there is inflammation at each puncture point, and there are different degrees of necrosis at canceration sites. It can be seen from a plurality of model pathological images that an injected liquid causes rapid interstitial necrosis, edema, fibrous tissue proliferation, paraneoplastic syndrome, and cancer tissue formation, and different degrees of necrosis at canceration sites can be observed during the cancer tissue formation; the injected liquid causes irritant necrosis; a process from liquid injection to cancer tissue formation is generally completed within 96 h; and after cancer tissue formation, a tumor tissue continues to grow. Therefore, in the present disclosure, rat and rabbit lung cancer animal models can be prepared by puncturing a body and injecting a variety of acids and a mixture of a saccharide and an acid into the body. It is well known that inflammation can appear in various tissues of a body, and a variety of acids and a mixture of a saccharide and an acid can enter various tissues of a body through a circulation system after being ingested. Therefore, the model is not limited to a lung cancer animal model, and can also be other various cancer tissue models. That is, the variety of acids and the mixture of a saccharide and an acid in the present disclosure can be used to prepare all cancer animal models including leukemia animal models.

In addition to preparation of cancer animal models, the composition of the present disclosure can be used to prepare animal models of other various human diseases. Because the long-term accumulation of an aldehyde compound in a body will reduce the permeability of a cell membrane, increase the transmembrane impedance, affect the normal metabolism of various cells, and lead to different tissue lesions. For example, when the aldehyde compound is attached to an outer membrane of a blood cell, necrotic blood cells will be deposited to cause thrombosis and CVDs; and when the aldehyde compound is attached to an outer membrane of a pancreatic β-cell, there will be a disorder in insulin secretion, thereby leading to diabetes. Thus, an organic acid or an inorganic acid may be used alone or in combination with glucose to prepare a variety of animal models of human diseases.

### BRIEF DESCRIPTION OF THE DRAWINGS

FIGS. 1A-IF show pathological staining results of rat lung tissues collected on day 14 after an experiment in a control group 1 in Experimental Example 1 (10×10), where FIG. 1A: no cancer, and mild inflammation; FIG. 1B: no cancer, and bleeding; FIG. 1C: no cancer, and mild inflammation; FIG. 1D: no cancer, and mild inflammation; FIG. 1E: no cancer, and mild inflammation; and FIG. 1F: no cancer, and mild inflammation;

FIGS. 2A-2E show pathological staining results of a control group 2 in Experimental Example 1 (10×10), where in an original experimental scheme, a sample is collected on day 14, rats are in a poor state after injection, and the sixth rat dies early, indicating that the formula cannot stably induce lung canceration in a healthy rat; a sample is collected at 36 h instead, and then an experiment is ended; and pathological analysis results of rats in this group are as follows: FIG. 2A: suspected cancer; FIG. 2B: no cancer; FIG. 2C: no cancer; FIG. 2D: no cancer; and FIG. 2E: no cancer;

FIGS. 3A-3F show pathological staining results of an experimental group 1 in Experimental Example 1 (10×10), where pathological analysis results at 96 h (4 d) after injection of an inducer are as follows: FIG. 3A: adenocarcinoma, few cancer glands; FIG. 3B: adenocarcinoma, a puncture point is at an apex of a lung; FIG. 3C: adenocarcinoma, few cancer glands; FIG. 3D: adenocarcinoma, many cancer glands; FIG. 3E: adenocarcinoma, many cancer glands; and FIG. 3F: adenocarcinoma, a superficial puncture point, pleural cancer;

FIGS. 4A-4F show pathological staining results of an experimental group 2 in Experimental Example 1 (10×10), where pathological analysis results at 168 h (7 d) after injection of an inducer are as follows: FIG. 4A: adenocarcinoma; FIG. 4B: adenocarcinoma; FIG. 4C: adenocarcinoma; FIG. 4D: adenocarcinoma; FIG. 4E: suspected adenocarcinoma, a depth of a puncture point is about 3 mm; and FIG. 4F: adenocarcinoma;

FIGS. 5A-5F show pathological staining results of an experimental group 3 in Experimental Example 1 (10×10), where pathological analysis results at 336 h (14 d) after injection of an inducer are as follows: FIG. 5A: adenocarcinoma, granulomatous inflammation; FIG. 5B: no cancer, granulomatous inflammation, a superficial puncture point; FIG. 5C: no cancer, a diameter of a puncture point is 2 mm; FIG. 5D: no cancer, no puncture point; FIG. 5E: adenocarcinoma, mild inflammation; and FIG. 5F: adenocarcinoma, no clear puncture point, severe intrapulmonary hemorrhage, submembrane cancer;

FIGS. 6A-6E show pathological staining results of an experimental group 1 in Experimental Example 2 (10×10), where a sample is collected on day 14 after an experiment; and pathological detection results of lung tissues are as follows: FIG. 6A: no cancer, moderate inflammation; FIG. 6B: no cancer, mild inflammation; FIG. 6C: no cancer, mild inflammation; FIG. 6D: no cancer, mild inflammation; and FIG. 6E: no cancer, mild inflammation;

FIGS. 7A-7E show pathological staining results of an experimental group 2 in Experimental Example 2 (10×10), where a sample is collected on day 7 after an experiment; and pathological detection results of lung tissues are as follows: FIG. 7A: adenocarcinoma; FIG. 7B: adenocarcinoma; FIG. 7C: adenocarcinoma; FIG. 7D: adenocarcinoma; and FIG. 7E: adenocarcinoma;

FIGS. 8A-8D show pathological staining results of an experimental group 3 in Experimental Example 2 (10×10), where a sample is collected on day 7 after an experiment; an animal dies at about 12 h, and a sample is not collected in time; and pathological detection results of lung tissues are as follows: FIG. 8A: adenocarcinoma; FIG. 8B: adenocarcinoma; FIG. 8C: no cancer, and alveolar epithelial dysplasia around a puncture point; and FIG. 8D: adenocarcinoma;

FIGS. 9A-9E show pathological staining results of an experimental group 4 in Experimental Example 2 (10×10), where a sample is collected on day 7 after an experiment; and pathological detection results of lung tissues are as follows: FIG. 9A: adenocarcinoma; FIG. 9B: adenocarcinoma; FIG. 9C: adenocarcinoma; FIG. 9D: adenocarcinoma; and FIG. 9E: no cancer, inflammatory secretions in a bronchial lumen, and severe inflammation;

FIGS. 10A-10E show pathological staining results of an experimental group 5 in Experimental Example 2 (10×10), where a sample is collected on day 7 after an experiment; and pathological detection results of lung tissues are as follows: FIG. 10A: adenocarcinoma; FIG. 10B: adenocarcinoma, tumor necrosis+a multinucleated giant cell (MGC) reaction; FIG. 10C: no cancer, severe inflammation; FIG. 10D: no cancer, mild inflammation, and no clear puncture point; and FIG. 10E: adenocarcinoma, and tumor cell necrosis;

FIGS. 11A-11D show pathological staining results of an experimental group 6 in Experimental Example 2 (10×10), where a sample is collected on day 7 after an experiment; an animal dies at about 16 h, and a sample is not collected in time: and pathological detection results of lung tissues are as follows: FIG. 11A: adenocarcinoma; FIG. 11B: no cancer, and no puncture point; FIG. 11C: adenocarcinoma; and FIG. 11D: adenocarcinoma;

FIGS. 12A-12E show pathological staining results of an experimental group 7 in Experimental Example 2 (10×10), where a sample is collected on day 7 after an experiment; and pathological detection results of lung tissues are as follows: FIG. 12A: no cancer, and a clear puncture point (an animal dies at 46 h); FIG. 12B: no cancer, and mild inflammation; FIG. 12C: adenocarcinoma; FIG. 12D: adenocarcinoma; and FIG. 12E: adenocarcinoma;

FIGS. 13A-13E show pathological staining results of an experimental group 8 in Experimental Example 2 (10×10), where a sample is collected on day 7 after an experiment; and pathological detection results of lung tissues are as follows: FIG. 13A: no cancer, mild inflammation, and no clear puncture point: FIG. 13B: adenocarcinoma; FIG. 13C: adenocarcinoma, and local alveolar epithelial dysplasia; FIG. 13D: no cancer, and local alveolar epithelial dysplasia; and FIG. 13E: adenocarcinoma;

FIGS. 14A-14E show pathological staining results of an experimental group 9 in Experimental Example 2 (10×10), where a sample is collected on day 7 after an experiment; and pathological detection results of lung tissues are as follows: FIG. 14A: adenocarcinoma; FIG. 14B: adenocarcinoma, diaphragmatic atrophy, and fibrous tissue proliferation; FIG. 14C: adenocarcinoma; FIG. 14D: adenocarcinoma; and FIG. 14E: adenocarcinoma; and

FIGS. 15A-15E show pathological staining results of an experimental group 10 in Experimental Example 2 (10×10), where a sample is collected on day 7 after an experiment; and pathological detection results of lung tissues are as follows: FIG. 15A: no cancer; FIG. 15B: adenocarcinoma; FIG. 15C: adenocarcinoma; FIG. 15D: adenocarcinoma; and FIG. 15E: adenocarcinoma, and tumor necrosis.

### DETAILED DESCRIPTION OF THE EMBODIMENTS

The present disclosure will be described in further detail below with reference to specific examples. The examples given are only for the purpose of illustrating the present disclosure, and are not intended to limit the scope of the present disclosure.

Unless otherwise specified, all experimental methods in the following examples are conventional methods. Unless otherwise specified, the medicinal raw materials, reagent materials, or the like used in the following examples all are commercially-available products. The experimental materials and sources thereof in the following examples are as follows: glucose (≥99.5%): Sinopharmn Chemical Reagent Co., Ltd., batch No.: 20140821; acetic acid (≥99.5%): Sinopharm Chemical Reagent Co., Ltd., batch No.: 20160920; and ethanol (≥95%): Sinopharm Chemical Reagent Co., Ltd., batch No.: 20200810.

### Example 1 the Composition of the Present Disclosure

Formula: glucose: 10 g, and acetic acid: 1 mL.
Preparation method: The 10 g of glucose and the 1 mL of acetic acid were thoroughly mixed for later use.

### Example 2 the Composition of the Present Disclosure

Formula: glucose: 10 g, and acetic acid: 2 mL.
Preparation method: The 10 g of glucose and the 2 mL of acetic acid were thoroughly mixed for later use.

### Example 3 the Composition of the Present Disclosure

Formula: glucose: 10 g, and acetic acid: 3 mL.
Preparation method: The 10 g of glucose and the 3 mL of acetic acid were thoroughly mixed for later use.

### Example 4 the Composition of the Present Disclosure

Formula: glucose: 10 g, and acetic acid: 4 mL.
Preparation method: The 10 g of glucose and the 4 mL of acetic acid were thoroughly mixed for later use.

### Example 5 the Composition of the Present Disclosure

Formula: glucose: 10 g, and acetic acid: 5 mL.
Preparation method: The 10 g of glucose and the 5 mL of acetic acid were thoroughly mixed for later use.

### Example 6 the Composition of the Present Disclosure

Formula: glucose: 10 g, and acetic acid: 6 mL.
Preparation method: The 10 g of glucose and the 6 mL of acetic acid were thoroughly mixed for later use.

### Example 7 the Composition of the Present Disclosure

Formula: glucose: 10 g, and acetic acid: 7 mL.
Preparation method: The 10 g of glucose and the 7 mL of acetic acid were thoroughly mixed for later use.

### Example 8 the Composition of the Present Disclosure

Formula: glucose: 10 g, and acetic acid: 8 mL.
Preparation method: The 10 g of glucose and the 8 mL of acetic acid were thoroughly mixed for later use.

### Example 9 the Composition of the Present Disclosure

Formula: glucose: 10 g, and acetic acid: 9 mL.
Preparation method: The 10 g of glucose and the 9 mL of acetic acid were thoroughly mixed for later use.

### Example 10 the Composition of the Present Disclosure

Formula: glucose: 10 g, and acetic acid: 10 mL.
Preparation method. The 10 g of glucose and the 10 mL of acetic acid were thoroughly mixed for later use.

### Example 11 the Composition of the Present Disclosure

Formula: glucose: 10 g, acetic acid: 1 mL, and ethanol: 1 mL.
Preparation method: The 10 g of glucose, the 1 mL of acetic acid, and the 1 mL of ethanol were thoroughly mixed for later use.

### Example 12 the Composition of the Present Disclosure

Formula: glucose: 10 g, acetic acid: 2 mL, and ethanol: 2 mL.
Preparation method: The 10 g of glucose, the 2 mL of acetic acid, and the 2 mL of ethanol were thoroughly mixed for later use.

### Example 13 the Composition of the Present Disclosure

Formula: glucose: 10 g, acetic acid: 3 mL, and ethanol: 3 mL.
Preparation method: The 10 g of glucose, the 3 mL of acetic acid, and the 3 mL of ethanol were thoroughly mixed for later use.

### Example 14 the Composition of the Present Disclosure

Formula: glucose: 10 g, acetic acid: 4 mL, and ethanol: 4 mL.
Preparation method: The 10 g of glucose, the 4 mL of acetic acid, and the 4 mL of ethanol were thoroughly mixed for later use.

### Example 15 the Composition of the Present Disclosure

Formula: glucose: 10 g, acetic acid: 5 mL, and ethanol: 5 mL.
Preparation method: The 10 g of glucose, the 5 mL of acetic acid, and the 5 mL of ethanol were thoroughly mixed for later use.

### Example 16 the Composition of the Present Disclosure

Formula: glucose: 10 g, acetic acid: 6 mL, and ethanol: 6 mL.
Preparation method: The 10 g of glucose, the 6 mL of acetic acid, and the 6 mL of ethanol were thoroughly mixed for later use.

### Example 17 the Composition of the Present Disclosure

Formula: glucose: 10 g, acetic acid: 7 mL, and ethanol: 7 mL.
Preparation method: The 10 g of glucose, the 7 mL of acetic acid, and the 7 mL of ethanol were thoroughly mixed for later use.

### Example 18 the Composition of the Present Disclosure

Formula: glucose: 10 g, acetic acid: 8 mL, and ethanol: 8 mL.
Preparation method: The 10 g of glucose, the 8 mL of acetic acid, and the 8 mL of ethanol were thoroughly mixed for later use.

### Example 19 the Composition of the Present Disclosure

Formula: glucose: 10 g, acetic acid: 9 mL, and ethanol: 9 mL.
Preparation method: The 10 g of glucose, the 9 mL of acetic acid, and the 9 mL of ethanol were thoroughly mixed for later use.

### Example 20 the Composition of the Present Disclosure

Formula: glucose: 10 g, acetic acid: 10 mL, and ethanol: 10 mL.
Preparation method: The 10 g of glucose, the 10 mL of acetic acid, and the 10 mL of ethanol were thoroughly mixed for later use.

Experimental Example 1 A study on induction of lung cancer in a rat by the composition of the present disclosure

### 1. Experimental Purpose

This experiment was intended to test and verify the induction of lung cancer in a rat model by the composition of the present disclosure at different time points.

### 2. Materials and Methods

### 2.1 Experimental Materials and Reagents

### 2.1.1 Main Experimental Reagents

| | |
|---|---|
| Sumianxin | Changsha Best Biological |
| | Technology Institute Co., Ltd. |
| Chloral hydrate | Aladdin Reagent (Shanghai) Co., Ltd. |
| Isoflurane | Shenzhen RWD Life Science Co., Ltd. |
| Xylene | Beijing Sinopharm Chemical |
| | Reagent Co., Ltd. |
| Absolute ethanol | Beijing Sinopharm Chemical |
| | Reagent Co., Ltd. |
| Hematoxylin and eosin | Germany Leica Biosystems |
| (H&E) staining kit | |
| Neutral formalin | Beijing Sinopharm Chemical |
| | Reagent Co., Ltd. |
| 1% hydrochloric acid | Germany Leica Biosystems |
| in an alcohol | |
| Neutral gum | Germany Leica Biosystems |
| 1% ammonia in water | Germany Leica Biosystems |
| Glucose | Sinopharm Chemical Reagent Co., Ltd. |
| 50% glucose injection | Henan Kelun Pharmaceutical Co., Ltd. |
| Concentrated hydrochloric | Sinopharm Chemical Reagent Co., Ltd. |
| acid (37% analytically pure) | Liaoning Minkang Pharmaceutical Co., Ltd. |
| NS (sodium chloride | |
| injection: 0.9%) | |

### 2.1.2 Main Experimental Devices

| | |
|---|---|
| Automatic advanced multispectral | American Perkin Elmer, model: |
| slide analysis system | N-MSI-vectra |
| Automatic histochemical | Germany Leica, model: ST5010 4169 |
| staining machine | |
| Manual rotary microtome | Germany Leica, model: RM2235 |
| Glass slide and cover slide | Jiangsu Citotest Scientific Co., Ltd. |
| Automatic dehydrator | Germany Leica, model: ASP200S 6162 |
| Hot stage of an | Germany Leica, model: |
| embedding machine | EG1150H 11323 |
| Cold stage of an | Germany Leica, model: |
| embedding machine | EG1150C 11498 |
| Freezing microtome | Germany Leica, model: CM1860UV |
| Spreading machine | Germany Leica, model: HI1210 |
| Slide drier | Germany Leica, model: HI1220 |
| Electronic balance | Shanghai Hochoice Industrial Co., |
| | Ltd., model: JE319 |
| PHS-3C PH meter | Shanghai INESA Scientific |
| | Instrument Co., Ltd. |

### 2.2 Experimental Animals

Species & strain: SD rats;
Animal grade: SPF grade;
Sex: male;
Source of experimental animals: Liaoning Changsheng Biotechnology Co., Ltd.;
Age or body weight: 200 g+20 g; and
Quantity: 30.

### 2.3 Experimental Method

### 2.3.1 Raise of Experimental Animals

The experimental animals were raised in a room with a temperature of 18° C. to 22° C., a humidity of 40% to 70%, an air change rate of no less than 20 times/h, no more than 6 animals in each cage, and a 12 h light/12 h dark alternating cycle. The animals were given a qualified rat feed, with free food intake. The animals were given purified water supplied by a drinking bottle, with free water intake.

### 2.3.2 Induction Injection

The grouping and administration of experimental animals were as follows:

According to body weights, the animals were randomly divided into 5 groups, with 6 animals per group. The administration and dose of an inducer for animals in each group were shown in the table below:

| | | Administration | | | Number of | Sampling | |
|---|---|---|---|---|---|---|---|
| Group | Modeling drug | mode | Position | Volume | animals (n) | time | |
| Control | 4 g of glucose is dissolved | Injection at a | Left | 50 uL | 6 | 14 | d |
| group 1 | in 1.5 mL of NS, and a | lung | lung | | | | |
| | resulting solution is heated | | | | | | |
| | to obtain a colorless | | | | | | |
| | transparent solution. | | | | | | |
| Control | 2.5 mL of NS is mixed with | Injection at a | Left | 50 uL | 6 | 36 | h |
| group 2 | 1.5 mL of hydrochloric acid. | lung | lung | | | | |
| Experimental | Inducer | Injection at a | Left | 50 uL | 6 | 4 | d |
| group 1 | | lung | lung | | | | |
| Experimental | Inducer | Injection at a | Left | 50 uL | 6 | 7 | d |
| group 2 | | lung | lung | | | | |
| Experimental | Inducer | Injection at a | Left | 50 uL | 6 | 14 | d |

| | | |
|---|---|---|
| group 3 | lung | lung |

Notes:
A preparation method of the inducer in the table above was as follows: 1 g of glucose was added to 0.375 mL of hydrochloric acid, and a resulting mixture was heated and stirred at a low temperature to obtain a colorless transparent viscous solution.

A rat was anesthetized with isoflurane, a left axilla of the rat was shaved to expose a skin, and the skin was sterilized with iodophor; 50 μL of a modeling drug was drawn with a microsyringe and injected into a lung of the rat, with the rat lying on its right side, a left forelimb naturally bent, an angle between a rib behind an elbow joint of the left forelimb of the rat and a horizontal plane being 45°, and a depth of 1 cm, and the generation of a lung cancer model was detected at different time points.

### 2.3.3 Pathological Detection

A rat was intravenously injected with chloral hydrate for anaesthetization and then sacrificed, and a chest cavity was opened; according to an administration site at a lung, a lesion at the administration site was observed, and a tissue was collected; and the tissue was fixed, subjected to programmed dehydration, embedded, sectioned, spread, dried, dewaxed in xylene for 5 min 3 times and then in 100% ethanol for 2 min 2 times, rinsed with tap water for 2 min, stained with hematoxylin for 3 min, rinsed with tap water for 2 min, differentiated with 1% hydrochloric acid in an alcohol for 2 s, rinsed with tap water for 2 min, stained with a blue-promoting solution for 1 s, rinsed with tap water for 2 min, stained with eosin for 10 s, dehydrated with 50% ethanol for 10 s, then with 70% ethanol for 10 s, and then with absolute ethanol for 1 min 2 times, and permeabilized in xylene for 3 min 2 times, then mounted with a neutral gum, and photographed by a microscope and observed.

### 2.4 Experimental Results

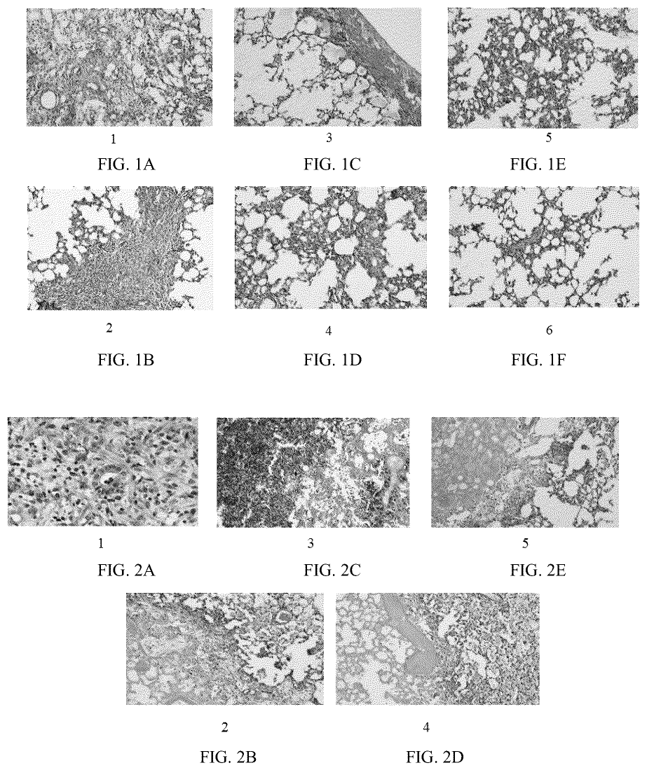

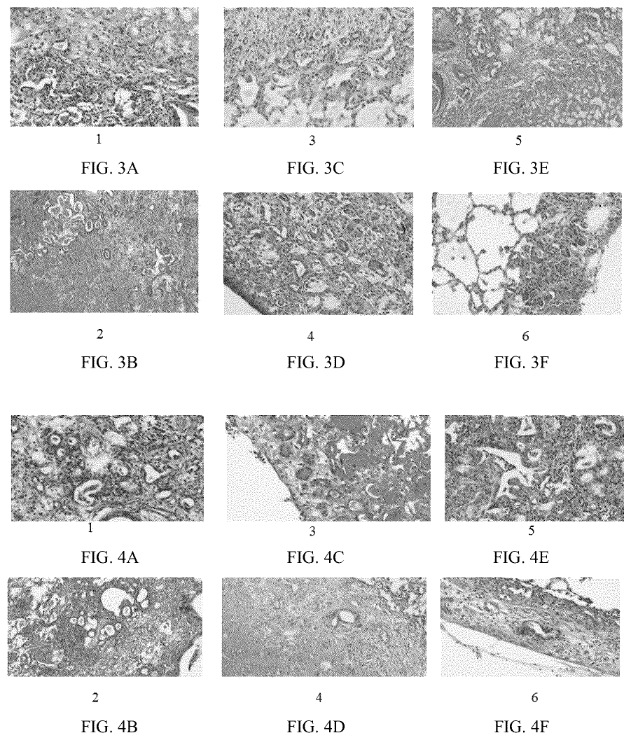

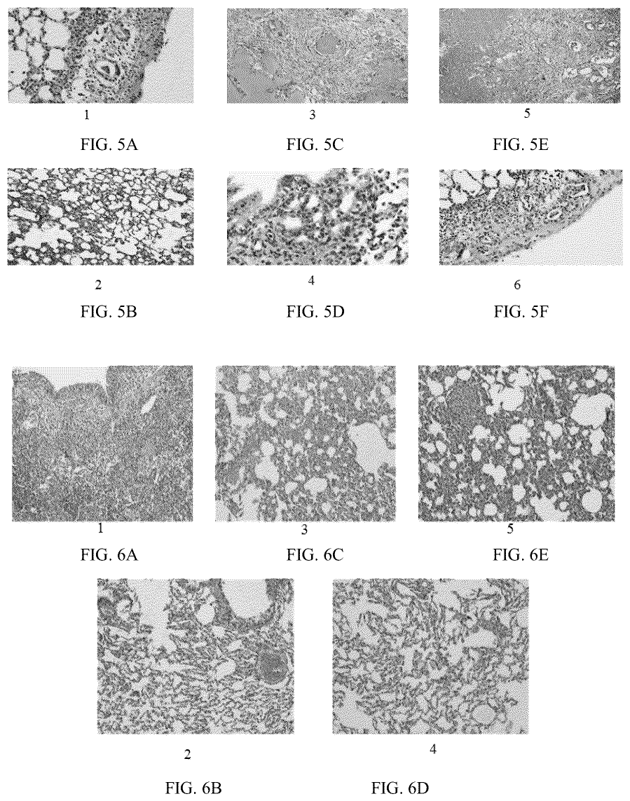

HE pathological staining results for each experimental group were shown in FIGS. 1A-1F to FIGS. 5A-5F.

### 3. Conclusion

It can be seen from this study that, in the control group 1, glucose and NS fail to induce carcinogenesis; in the control group 2, NS and hydrochloric acid have strong toxicities and fail to clearly induce carcinogenesis in a short period of time; and after the formula of the inducer is optimized in the experimental group, carcinogenesis is detected in an injection zone on day 4, and pathological detection results on day 7 and day 14 further confirm an effect of the inducer for inducing adenocarcinoma carcinogenesis in a lung of a rat, indicating that the inducer prepared in this way can induce carcinogenesis at a lung of a rat in a short term.

Experimental Example 2 A study on induction of carcinogenesis in a lung tissue of a rabbit by the composition of the present disclosure

### 1. Experimental Purpose

This experiment was intended to test and verify the induction of lung cancer in an experimental rabbit model by the composition of the present disclosure at different time points.

### 2. Materials and Methods

### 2.1 Experimental Materials and Reagents

### 2.1.1 Main Experimental Reagents

| | |
|---|---|
| Sumianxin | Changsha Best Biological Technology |
| | Institute Co., Ltd. |
| Chloral hydrate | Aladdin Reagent (Shanghai) Co., Ltd. |
| Isoflurane | Shenzhen RWD Life Science Co., Ltd. |
| Xylene | Beijing Sinopharm Chemical |
| | Reagent Co., Ltd. |
| Absolute ethanol | Beijing Sinopharm Chemical |
| | Reagent Co., Ltd. |
| Hematoxylin and eosin | Germany Leica Biosystems |
| (H&E) staining kit | |
| Neutral formalin | Beijing Sinopharm Chemical |
| | Reagent Co., Ltd. |
| 1% hydrochloric acid | Germany Leica Biosystems |
| in an alcohol | |
| Neutral gum | Germany Leica Biosystems |
| 1% ammonia in water | Germany Leica Biosystems |
| Glucose | Sinopharm Chemical Reagent Co., Ltd. |
| 50% glucose injection | Henan Kelun Pharmaceutical Co., Ltd. |
| Concentrated hydrochloric acid | Sinopharm Chemical Reagent Co., Ltd. |
| (37% analytically pure) | Liaoning Minkang |
| | Pharmaceutical Co., Ltd. |
| NS (sodium chloride | Sinopharm Chemical Reagent Co., Ltd. |
| injection: 0.9%) | |
| Acetic acid (analytically pure) | Sinopharm Chemical Reagent Co., Ltd. |
| Phosphoric acid | Sinopharm Chemical Reagent Co., Ltd. |
| (analytically pure) | |
| Lactic acid (analytically pure) | |

### 2.1.2 Main Experimental Devices

| | |
|---|---|
| Automatic advanced multispectral | American Perkin Elmer, |
| slide analysis system | model: N-MSI-vectra |
| Automatic histochemical | Germany Leica, model: ST5010 4169 |
| staining machine | |
| Manual rotary microtome | Germany Leica, model: RM2235 |
| Glass slide and cover slide | Jiangsu Citotest Scientific Co., Ltd. |
| Automatic dehydrator | Germany Leica, model: ASP200S 6162 |
| Hot stage of an | Germany Leica, model: |
| embedding machine | EG1150H 11323 |
| Cold stage of an | Germany Leica, model: |
| embedding machine | EG1150C 11498 |
| Freezing microtome | Germany Leica, model: CM1860UV |
| Spreading machine | Germany Leica, model: HI1210 |
| Slide drier | Germany Leica, model: HI1220 |
| Electronic balance | Shanghai Hochoice Industrial |
| | Co., Ltd., model: JE319 |
| PHS-3C pH meter | Shanghai INESA Scientific |
| | Instrument Co., Ltd. |

### 2.2 Experimental Animals

Species & strain: New Zealand white rabbits;
Animal grade: SPF grade;
Sex: male;
Source of experimental animals: National Institutes for Food and Drug Control;
Age or body weight: 1.8 kg to 2.2 kg; and
Quantity: 50.

### 2.3 Experimental Method

### 2.3.1 Raise of Experimental Animals

The experimental animals were raised in a room with a temperature of 18° C. to 22° C., a humidity of 40% to 70%, an air change rate of no less than 20 times/h, no more than 2 animals in each cage, and a 12 h light/12 h dark alternating cycle. The animals were given a qualified feed, with free food intake. The animals were given purified water supplied by a drinking bottle, with free water intake.

### 2.3.2 Induction Injection

A model was prepared as follows: According to body weights, the animals were randomly divided into 10 groups, with 5 animals per group. The administration and dose of an inducer for animals in each group were shown in the table below:

| | | | | | Number | | | |
|---|---|---|---|---|---|---|---|---|
| | | | | | of | | | |
| | | Administration | | | animals | Detection | | |
| Group | Modeling drug | mode | Position | Volume | (n) | time | | pH |
| Experimental | A 50% glucose | Injection at a | Left lung | 600 uL | 5 | 14 | d | 3.22 |
| group 1 | injection is mixed | lung | | | | | | |
| (control | with NS (volume | | | | | | | |
| group) | ratio: 10:1). | | | | | | | |
| Experimental | NS is mixed with | Injection at a | Left lung | 100 uL | 5 | 7 | d | 0 |
| group 2 | concentrated | lung | | | | | | |
| | bydrochloric acid | | | | | | | |
| | (volume ratio: 5:3). | | | | | | | |
| Experimental | NS is mixed with | Injection at a | Left lung | 300 uL | 5 | 7 | d | 2.20 |
| group 3 | acetic acid (volume | lung | | | | | | |
| | ratio: 10:1). | | | | | | | |
| Experimental | NS is mixed with | Injection at a | Left lung | 300 uL | 5 | 7 | d | 1.83 |
| group 4 | lactic acid (volume | lung | | | | | | |
| | ratio: 10:1). | | | | | | | |
| Experimental | NS is mixed with | Injection at a | Left lung | 300 uL | 5 | 7 | d | 1.31 |
| group 5 | phosphoric acid | lung | | | | | | |
| | (volume ratio: 10:1). | | | | | | | |
| Experimental | 1 g of glucose is | Injection at a | Left lung | 300 uL | 5 | 7 | d | 0 |
| group 6 | added to 0.375 mL of | lung | | | | | | |
| | analytically-pure | | | | | | | |
| | hydrochloric acid, | | | | | | | |
| | and resulting | | | | | | | |
| | mixture is heated and | | | | | | | |
| | stirred at a low | | | | | | | |
| | temperature to obtain | | | | | | | |
| | 3 colorless transparent | | | | | | | |
| | viscous solution. | | | | | | | |
| Experimental | A 50% glucose | Injection at a | Left lung | 300 uL | 5 | 7 | d | 2.08 |
| group 7 | injection is mixed | lung | | | | | | |
| | with acetic acid | | | | | | | |
| | (volume ratio: 10:1). | | | | | | | |
| Experimental | A 50% glucose | Injection at a | Left lung | 300 uL | 5 | 7 | d | 2.17 |
| group 8 | injection is mixed | lung | | | | | | |
| | with acetic acid | | | | | | | |
| | (volume ratio: 20:1). | | | | | | | |
| Experimental | A 50% glucose | Injection at a | Left lung | 600 uL | 5 | 7 | d | 1.72 |
| group 9 | injection is mixed | lung | | | | | | |
| | with lactic acid | | | | | | | |
| | (volume ratio: 10:1). | | | | | | | |
| Experimental | A 50% glucose | | | | | | | |
| group 10 | injection is mixed | Injection at a | Left lung | 600 uL | 5 | 7 | d | 1.36 |

| | |
|---|---|
| with phosphoric acid | lung |
| (volume ratio: 10:1). | |

An experimental rabbit was fixed, with the experimental rabbit lying on its right side; a left axilla of the experimental rabbit was shaved to expose the skin, and the skin was sterilized with iodophor; and 300 μL of a modeling drug was drawn with 1 mL syringe and completely injected into a lung of the experimental rabbit at a position that was between the 7th and 8th ribs counting upwards from a lower edge of ribs and was at a 2-finger-width distance from a spine, with a depth of about 2.5 cm.

### 2.3.3 Pathological Detection

An experimental rabbit was intravenously injected with chloral hydrate for anaesthetization and then sacrificed, and a chest cavity was opened: according to an administration site at a lung, a lesion at the administration site was observed, and a tissue was collected; and the tissue was fixed, subjected to programmed dehydration, embedded, sectioned, spread, dried, dewaxed in xylene for 5 min 3 times and then in 100% ethanol for 2 min 2 times, rinsed with tap water for 2 min, stained with hematoxylin for 3 min, rinsed with tap water for 2 min, differentiated with 1% hydrochloric acid in an alcohol for 2 s, rinsed with tap water for 2 min, stained with a blue-promoting solution for 1 s, rinsed with tap water for 2 min, stained with eosin for 10 s, dehydrated with 50% ethanol for 10 s, then with 70% ethanol for 10 s, and then with absolute ethanol for 1 min 2 times, and permeabilized in xylene for 3 min 2 times, then mounted with a neutral gum, and photographed by a microscope and observed.

### 2.4 Experimental Results

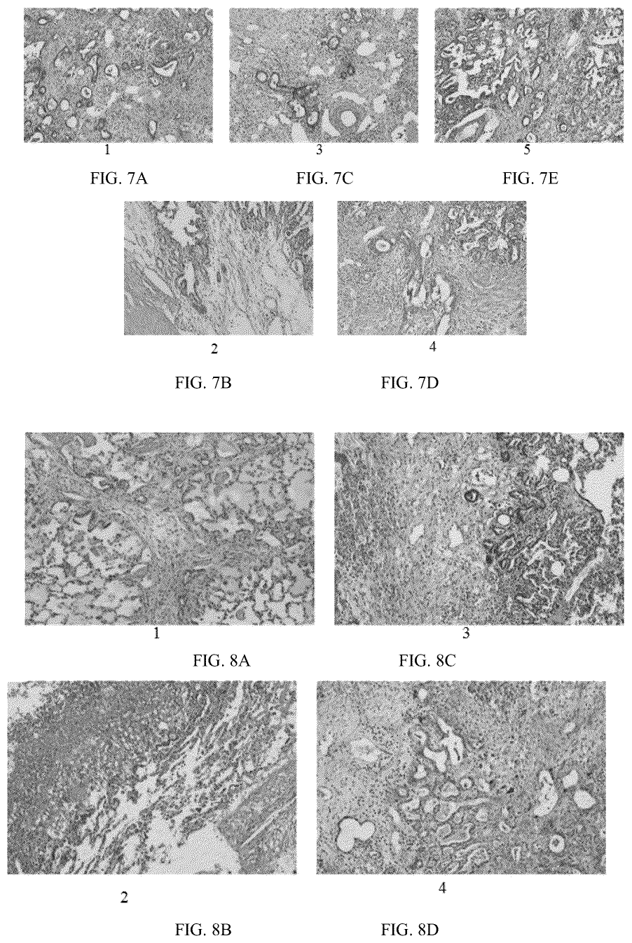

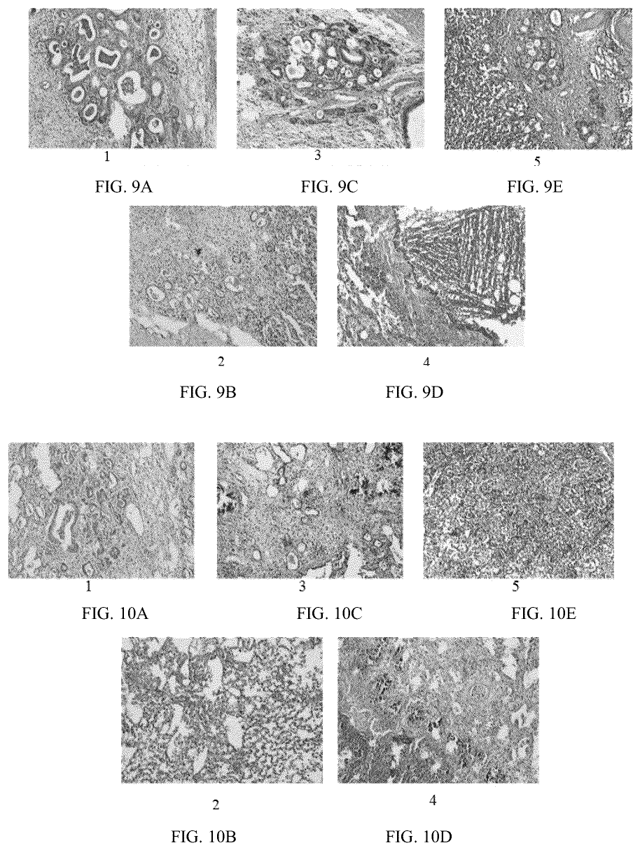

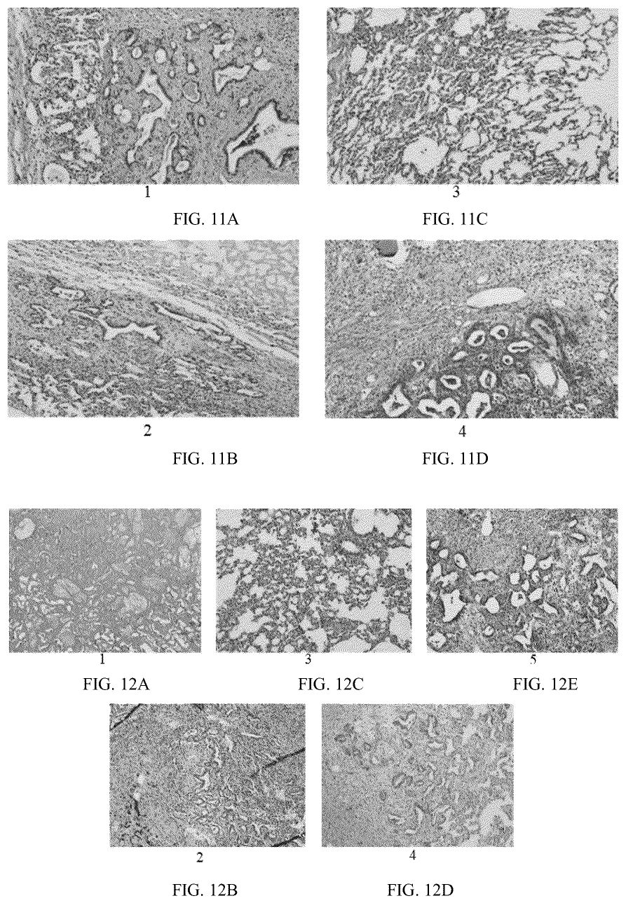

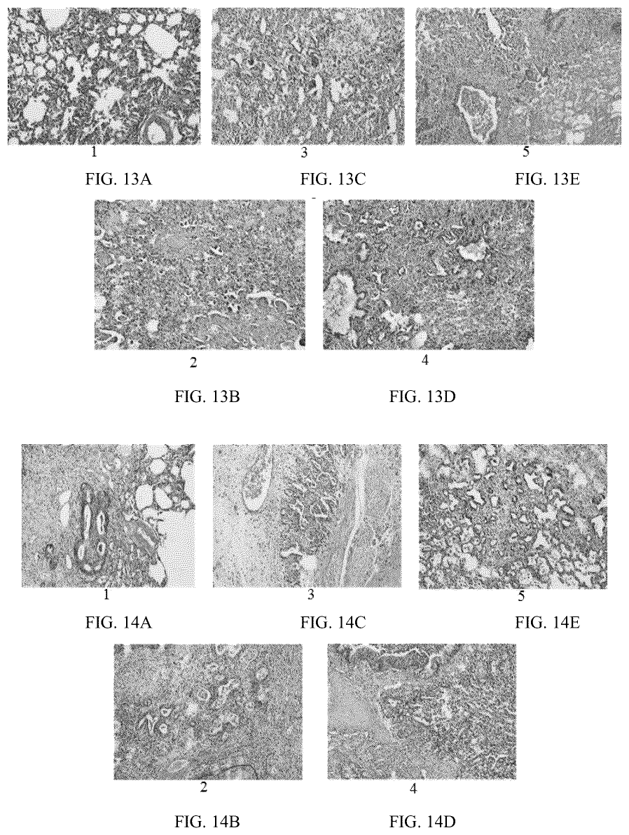

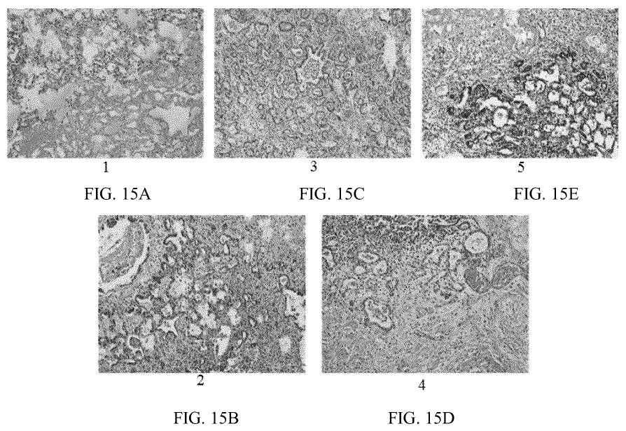

HE pathological staining results for each experimental group were shown in FIGS. 6A-6E to FIGS. 15A-15E.

### 3. Conclusion

It can be seen from this study that, except for the experimental group 1 (control group), the other groups all can induce carcinogenesis within 7 d, and consider both safety and induction efficiency; the formulas of the experimental group 2 (NS and concentrated hydrochloric acid (volume ratio: 5:3)) and the experimental group 9 (50% glucose injection and lactic acid (volume ratio: 10:1)) are the optimal; and the addition of glucose to the formula can effectively reduce a degree of local acute stimulation and reduce the death of experimental animals.

What is claimed is:

1. A composition, comprising a saccharide and an acid.

2. The composition according to claim 1, wherein the saccharide is a polyhydroxy (two or more hydroxy groups) aldehyde or ketone compound, the polyhydroxy aldehyde or ketone compound comprises two or more hydroxy groups, and the saccharide is composed of carbon, hydrogen, and oxygen elements;
the acid is an organic acid or an inorganic acid;
a mass-to-volume ratio of the saccharide to the acid is 1:100 to 1001 (g/mL);
the composition has a pH of lower than 4; and
the composition is a food composition.

3. A composition, comprising a saccharide, an acid, and an alcohol.

4. The composition according to claim 3, wherein the alcohol is ethanol;
the saccharide, the acid, and the alcohol are in a ratio of 1:100:1 to 100:1:100 (g/mL/mL); and
the composition is a food composition.

5. A method of using the composition according to claim 1 in a preparation of an animal model.

6. The method according to claim 5, wherein a model animal used for the animal model comprises a mammal, wherein the mammal is a mouse, a rat, a rabbit, a dog, a monkey, a cat, a chicken, or a pig;
the animal model is prepared by administering the composition to the model animal through an administration route, wherein the administration route is a feeding, an intragastric administration, an intravenous drip, an_intravenous injection, or a skin penetration; and
the animal model comprises an animal model of a diabetes, a cancer, an atherosclerosis (AS) or a CVD caused thereby.

7. The method according to claim 5, wherein the animal model is used in a drug screening for a prevention and/or a treatment of the cancer, the diabetes, the AS or the CVD caused thereby.

8. A method of using an acid in a preparation of an animal model, wherein the acid is an inorganic acid or an organic acid;
and
the acid has a pH of lower than 4.

9. The method according to claim 8, wherein a model animal used for the animal model comprises a mammal, wherein the mammal is a mouse, a rat, a rabbit, a dog, a monkey, a cat, a chicken, or a pig;
the animal model is prepared by administering the acid to the model animal through an administration route, wherein the administration route is a feeding, an intragastric administration, an intravenous drip, an intravenous injection, or a skin penetration; and
the animal model comprises an animal model of a diabetes, a cancer, an AS or a CVD caused thereby.

10. The method use-according to claim 8, wherein the animal model is used in a drug screening for a prevention and/or a treatment of the cancer, the diabetes, the AS or the CVD caused thereby.

11. A modeling method, comprising: administering the composition or the acid according to claim 1 to a model animal,
wherein the model animal comprises a mammal, wherein the mammal is a mouse, a rat, a rabbit, a dog, a monkey, a cat, a chicken, or a pig;
a prepared model comprises an animal model of a diabetes, a cancer, an AS or a CVD caused thereby;
the acid is an inorganic acid or an organic acid;
the acid is selected from the group consisting of 13.88% (mass percentage concentration) hydrochloric acid, 7.73% (mass percentage concentration) lactic acid, 4.74% to 9.05% (mass percentage concentration) acetic acid, and 7.73% (mass percentage concentration) phosphoric acid; and
the acid has a pH of lower than 4.

12. The composition according to claim 1, wherein
the saccharide is glucose;
the acid is selected from the group consisting of 85% (mass percentage concentration) phosphoric acid, 37% (mass percentage concentration) hydrochloric acid, 99.5% (mass percentage concentration) acetic acid, and 85% (mass percentage concentration) lactic acid;
the mass-to-volume ratio of the saccharide to the acid is 1:20 to 20:1 (g/mL); and
the composition has the pH of lower than 3.22.

13. The composition according to claim 1, wherein the mass-to-volume ratio of the saccharide to the acid is 1:0.375 (g/mL) or 5:1 (g/mL).

14. The composition according to claim 3, wherein the saccharide, the acid, and the alcohol are in the ratio of 1:20:1 to 20:1:20 (g/mL/mL).

15. The composition according to claim 3, wherein the saccharide, the acid, and the alcohol are in the ratio of 10:5:5 (g/mL/mL).

16. The method according to claim 5, wherein in the composition, the saccharide is a polyhydroxy aldehyde or ketone compound, the polyhydroxy aldehyde or ketone compound comprises two or more hydroxyl groups, and the saccharide is composed of carbon, hydrogen, and oxygen elements;
the acid is an organic acid or an inorganic acid;
a mass-to-volume ratio of the saccharide to the acid is 1:100 to 100:1 (g/mL);
the composition has a pH of lower than 4; and
the composition is a food composition.

17. A method of using the composition according to claim 3 in a preparation of an animal model.

18. The method according to claim 17, wherein the alcohol is ethanol;
the saccharide, the acid, and the alcohol are in a ratio of 1:100:1 to 100:1:100 (g/mL/mL); and
the composition is a food composition.

19. The method according to claim 8, wherein
the acid is selected from the group consisting of 13.88% (mass percentage concentration) hydrochloric acid, 7.73% (mass percentage concentration) lactic acid, 4.74% to 9.05% (mass percentage concentration) acetic acid, and 7.73% (mass percentage concentration) phosphoric acid; and
the acid has the pH of lower than 3.22.

20. The modeling method according to claim 11, wherein in the composition, the saccharide is a polyhydroxy aldehyde or ketone compound, the polyhydroxy aldehyde or ketone compound comprises two or more hydroxyl groups, and the saccharide is composed of carbon, hydrogen, and oxygen elements;
the acid is an organic acid or an inorganic acid;
a mass-to-volume ratio of the saccharide to the acid is 1:100 to 100:1 (g/mL);
the composition has a pH of lower than 4; and
the composition is a food composition.
